(19) United States
(12) Patent Application Publication
(10) Pub. No.: US 2014/0350129 A1
(43) Pub. Date: Nov. 27, 2014

(54) DIAGNOSTIC ASSAY TO PREDICT CARDIOVASCULAR RISK

(71) Applicants: Sergey Sikora, San Diego, CA (US); Stephen Epstein, Hyattsville, MD (US); Arshed A. Quyyumi, Atlanta, GA (US)

(72) Inventors: Sergey Sikora, San Diego, CA (US); Stephen Epstein, Hyattsville, MD (US); Arshed A. Quyyumi, Atlanta, GA (US)

(73) Assignees: Genway Biotech, Inc., San Diego, CA (US); Emory University, Atlanta, GA (US); Medstar Health Research Institute, Hyattsville, MD (US)

(21) Appl. No.: 14/342,522

(22) PCT Filed: Mar. 1, 2012
(86) PCT No.: PCT/US2012/027354

§ 371 Date: Aug. 5, 2014

Related U.S. Application Data
(60) Provisional application No. 61/532,012, filed on Sep. 7, 2011.

(51) Int. Cl. G01N 33/68; G01N 33/86

(52) U.S. Cl. CPC G01N 33/6893; G01N 33/86; G01N 2800/324; G01N 2800/325; G01N 2800/50; G01N 2800/52; G01N 2333/75; G01N 2333/4737
USPC 514/789; 506/9

(57) ABSTRACT

This invention relates to the area of cardiovascular disorders and specifically relates to methods of diagnostic tests using a combination of markers to predict an individual's risk for developing coronary artery disease (CAD) and related diseases, such as angina pectoris and peripheral vascular disease and, more particularly, to determine an individual's risk of myocardial infarction, death, and stroke. Exemplary biomarkers include C-reactive protein (CRP), fibrin degradation products (FDPs), Heat Shock Protein 70 (HSP70), and/or anti-CMV antibody.

### CROSS-REFERENCE TO RELATED APPLICATIONS

This application claims priority from U.S. provisional application No. 61/532,012, filed Sep. 7, 2011, the contents of which are incorporated by reference in their entirety.

### TECHNICAL FIELD

The invention in some aspects relates to diagnostic and predictive tests in which a combination of markers is used to predict an individual's risk for (provide the likelihood of an individual's) developing coronary artery disease (CAD) and related diseases, such as angina pectoris and peripheral vascular disease and, more particularly, to determine an individual's risk of myocardial infarction, death, and/or stroke. The methods and compositions can be used in some aspects to 1) detect CAD or the risk of developing CAD in an individual in the absence of known coronary artery disease, 2) detect risk of acute myocardial infarction (AMI) or death in individuals with known CAD, and 3) to detect risk of AMI or death in individuals in the absence of known CAD.

### BACKGROUND

Coronary artery disease and its consequences affect millions of people worldwide; it is the leading cause of death in the United States. The most common manifestation of CAD is chest pain (angina pectoris) due to myocardial ischemia, which can lead to heart attack (acute myocardial infarction or AMI) and sudden death. According to the CDC, about 1.3 million Americans have a heart attack each year. In addition to those who exhibit clinical symptoms of ischemic heart disease, many other individuals are at high risk of developing heart disease based on indicators such as hypertension, high levels of serum cholesterol and/or family history. However, a national study from UCLA shows that approximately 75% of patients who had heart attacks did not have high cholesterol (Am Heart J. 2009 January; 157(1):111-117.e2. Epub 2008 Oct. 22). Of those patients who have heart attacks, 88% would have been deemed low to intermediate risk just the day before (BaleDoneen CVD prevention program. www.baledoneen.com). Current tools are remiss in determining many patients at high risk for cardiac events.

Current diagnostic procedures have not been entirely satisfactory, for example in identifying individuals at-risk for certain outcomes. Diagnostic and predictive methods and systems are needed for coronary artery disease, such as those that are non-invasive, sensitive, and reliable for the assessment and prediction of adverse cardiovascular outcomes, particularly for people at risk for CAD and its consequences. Provided are embodiments to meet these needs.

### SUMMARY

In one aspect, the invention described herein meets the need for assessing and predicting adverse cardiovascular outcomes, particularly for individuals at risk for CAD and individuals who have CAD and consequences of CAD. Provided are diagnostic and predictive methods for assessing, detecting, predicting, and prognosing adverse cardiovascular outcomes (and risks thereof), such as CAD and associated outcomes and conditions; systems, e.g. kits, for performing the methods; and methods of treatment, for example, of individuals assessed using the diagnostic or predictive methods. In some embodiments, the methods determine risk or assess increased probability of one or more adverse outcomes of CAD. In one aspect, the provided methods and systems meet the need for a noninvasive method for 1) detecting CAD or risk of developing CAD in an individual in the absence of known CAD, 2) detecting and predicting risk of myocardial infarction (MI), e.g., acute MI (AMI) and/or death, and predicting risk of stroke in a patient (individual) with known CAD, and 3) predicting risk of AMI and/or death and/or predicting risk of stroke in a patient (individual) with previously unknown CAD.

In some embodiments, the methods are carried out by detecting or measuring the presence, absence, expression, and/or level of biomarkers, typically of each of a plurality of biomarkers, in a sample, e.g., a test biological sample, such as one obtained from a subject being evaluated by the methods.

In some embodiments, the methods are carried out by contacting a test biological sample, such as one from the subject being evaluated by the methods, with a panel of agents that specifically bind to a plurality of biomarkers, thereby measuring levels of the plurality of biomarkers.

In some embodiments, the methods are carried out by comparing the level of each of the plurality of biomarkers in the subject, e.g., in a test sample from the subject, to a control level of the respective biomarker.

The plurality of biomarkers generally includes one or more of a thrombosis biomarker, a cellular stress biomarker, an inflammation biomarker, and/or an autoimmune biomarker, typically at least two of a thrombosis biomarker, a cellular stress biomarker or autoimmune biomarker, an inflammation biomarker, and in some aspects includes a thrombosis biomarker, a cellular stress biomarker or autoimmune biomarker, and an inflammation biomarker, for example, a thrombosis biomarker, a cellular stress biomarker, and an inflammation biomarker or a thrombosis biomarker, an autoimmune biomarker, and an inflammation biomarker. The plurality of biomarkers in some cases further includes an infection biomarker. The level of two or more, three or more or four (all) of the types of biomarkers (inflammatory biomarkers, infectious biomarkers, thrombotic biomarkers, cellular stress biomarkers, autoimmune biomarkers) can be assessed. The level of two or more biomarkers of the same type (e.g., two or more inflammatory biomarkers, two or more infectious biomarkers, two or more thrombotic biomarkers, two or more cellular stress biomarkers, two or more autoimmune biomarkers) can be measured.

In some embodiments, the thrombosis biomarker is an FDP marker, where the FDP marker includes at least one fibrin and fibrinogen degradation product (FDP), and in some embodiments includes a mixture of at least two fibrin and fibrinogen degradation products (FDPs). Thus, in some examples, the plurality of biomarkers includes an FDP marker, where the FDP marker includes at least one or a mixture of at least two fibrin and fibrinogen degradation product gene products, e.g., at least one or a mixture of at least two fibrin and fibrinogen degradation products (FDPs). In one example, the at least one or at least two FDPs include an FDP selected from among fragment D, fragment E, and D-dimer, or from among fragment D and fragment E. In one example, the at least one or two FDPs include fragment D, fragment E, and D-dimer, or fragment D and E. In some examples, the FDPs further include fragment X, fragment Y, or one or more initial plasmin digest products (IPDPs). In some cases, the at least one or two FDPs include one, more, or all FDPs detected by the DR-70® ELISA assay. In another example, they include one or more FDPs described in International Application Publication Number WO 2010/114514 A1. In another example they include one or more FDPs detected by a Fibrinogen ELISA assay.

Thus, in some embodiments, the panel of agents includes an agent or agents for detection of the FDP marker. In some aspects, the panel includes an agent or agents that is or are capable of specifically binding to at least two FDPs, such as at least two FDPs selected from among fragment D, fragment E, and D-dimer, or from among fragment D and fragment E. In one example, the at least one or two FDPs include fragment D, fragment E, and D-dimer, or fragment D and E. In some examples, the FDPs further include fragment X, fragment Y, or one or more initial initial plasmin digest products (IPDPs). In some cases, the at least one or two FDPs include one, more, or all FDPs detected by the DR-70® ELISA assay. In another example, they include one or more FDPs described in International Application Publication Number WO 2010/114514 A1. In another example they include one or more FDPs detected by a Fibrinogen ELISA assay.

In some such examples, the plurality of biomarkers further includes at least one inflammation or autoimmune disease or cellular stress biomarker, or at least one each of both an inflammation and an autoimmune disease or cellular stress biomarker, such as an inflammation and an autoimmune disease biomarker or an inflammation and a cellular stress biomarker. Thus, in some aspects, the agents include an agent or agent that specifically binds or bind to such biomarkers.

In some aspects, the at least one inflammation biomarker includes C-reactive protein (CRP) gene product, such as a CRP protein. In some aspects, the at least one autoimmune disease or cellular stress biomarker includes a Heat Shock Protein 70 (HSP70) gene product, such as an HSP70 protein. Thus, in some aspects, the agents include an agent or agent that specifically binds or bind to such biomarkers.

In some aspects, the biomarkers further include an anti-cytomegalovirus antibody gene product, such as an anti-cytomegalovirus antibody. In some aspects, the biomarkers further include an antibody to Heat Shock Protein 60 (anti-HSP60) gene product, e.g., an anti-HSP60 protein. Thus, in some aspects, the agents include an agent or agent that specifically binds or bind to such biomarkers.

In some embodiments, the plurality of biomarkers includes Heat Shock Protein 70 (HSP70), C-reactive protein (CRP), and an FDP marker. In some embodiments, the method measures levels of CRP and FDP marker, measures levels of CRP, FDP marker and HSP70, measures levels of CRP, FDP marker and anti-CMV Ab, or measures levels of CRP, FDP marker, HSP 70 and anti-CMV Ab. Thus, in some aspects, the agents include an agent or agent that specifically binds or bind to such biomarkers.

The levels, e.g., the detected or measured levels, or the levels relative to other levels, such as control levels, of the plurality of biomarkers generally indicate a risk of an adverse cardiovascular outcome in the subject. Typically, the levels, for example, the levels, in the aggregate, indicate the risk. For example, the levels of the biomarkers, in the aggregate, can indicate an increased risk of an adverse cardiovascular outcome, for example, that the adverse cardiovascular outcome is more likely in the subject compared to the average likelihood of the outcome in a given population of subjects, such as healthy individuals, individuals who do not have CAD, or individuals who are at low risk for such an outcome.

In some aspects, an elevated level or increased level of only one of the biomarkers, alone (i.e., without at least one another of the biomarkers elevated or increased) would not indicate the increased risk.

In some aspects, the levels (such as elevated levels), e.g., in the aggregate, of the biomarkers indicate at least a 1.5, 2, 2.5, 3, 3.5, 4, 4.5, 5, 5.5, 6, 6.5, 7, 8, 9, 10 or more times greater risk of the adverse cardiovascular outcome, such as stroke, or death, for example, annually, in the subject, compared to another subject, such as one in which none of the plurality of biomarkers is elevated or positive.

In some aspects, the determination that one biomarker, and typically at least two (e.g., 2, 3, 4, or more) biomarkers, is or are elevated indicates a risk of the adverse cardiovascular outcome in the subject, such as at or about or at least at or about 20%, 25%, 30%, 35%, 40%, 45%, 50%, 55%, 60%, 65%, 70%, or more risk of developing the adverse outcome, such as AMI or death, for example, within a given period of time, such as 1, 2, 3, 4, or more years, such as within 1200 days. In one example, determination that at least two biomarkers are elevated indicates an at or about 20%, 25%, 30%, or 35% risk of the adverse outcome within the period of time; in another example, determination that at least three biomarkers are elevated indicates an at or about 30%, 35%, 40%, 45%, or 50% risk of the adverse outcome within the period of time.

In one aspect, an increase in the levels of the plurality of biomarkers, for example, in the aggregate, in the subject, e.g., in the test sample compared to the control levels, indicates a risk of an adverse cardiovascular outcome in the subject.

In some embodiments, the level of the FDP marker is elevated or positive if greater than 1 micrograms per milliliter of sample; in some embodiments, the level of the CRP gene product is elevated or positive if greater than 3 milligrams per milliliter of sample; in one embodiment, the level of HSP70 gene product is elevated or positive if detectable in the sample.

As described herein, an aggregate score based on measures of CRP, FDP marker and HSP70 is a strong predictor of future risk of developing (the likelihood of developing) CAD in individuals. In one embodiment, the aggregate score is a strong predictor of risk of adverse effects of CAD, such as myocardial infarction (MI) (aka acute MI (AMI)) and death, in subjects not known to have CAD (e.g., patients being considered for assessment, such as by coronary angiography). In an alternative embodiment, the aggregate score is a strong predictor of risk of adverse effects of CAD, such as AMI and death, in subjects (e.g., patients) with known CAD.

Thus, the adverse cardiovascular outcome can be developing CAD, an adverse effect of CAD, such AMI, stroke, or death. In some cases, the adverse cardiovascular outcome is the occurrence of such an outcome in a given period of time, e.g., 1, 2, 3, 4, 5, or more years, such as within 1-2, 2-3, or 1-3 years. Thus, in some cases, the method predicts the near-term risk of any of the above outcomes, such as the risk that the outcome will occur in 2-3 years.

In the embodiments in which the level of an FDP marker is measured in a biological (test) sample from an individual being assessed, the individual being assessed is at increased risk or there is a greater probability of developing CAD or adverse outcomes of CAD if the level of the FDP marker in the biological sample is significantly higher than the level in a reference or standard, such as the FDP marker level in individuals who do not have CAD. In the embodiments in which the level of FDP marker and the level of CRP are measured in a biological sample from an individual being assessed, the individual being assessed is at increased risk or there is a greater probability of developing CAD or adverse outcomes of CAD if the level of FDP marker in the biological sample is significantly higher than the FDP marker level in a reference or standard, such as the FDP marker level in individuals who do not have CAD and if the level of CRP in the biological sample is significantly higher than the CRP level in a reference or standard, such as the CRP level in individuals who do not have CAD. In the embodiments in which the level of FDP marker, the level of CRP, and the level of HSP70 are measured in a biological sample from an individual being assessed, the individual being assessed is at increased risk or there is a greater probability of developing CAD or adverse outcomes of CAD if (a) the level of FDP marker in the biological sample is significantly higher than the FDP level in a reference or standard, such as the FDP marker level in individuals who do not have CAD; (b) the level of CRP in the biological sample is significantly higher than the CRP level in a reference or standard, such as the CRP level in individuals who do not have CAD and (c) HSP70 is significantly higher than the HSP70 level in a reference or standard, such as the HSP70 level in individuals who do not have CAD.

In one embodiment, if the level of FDP marker in the biological sample is determined to be greater than about 1.0 μg/ml; the level of CRP in the biological sample is greater than about 3.0 mg/L and HSP70 is detectable, for example, even if it is detected at very low levels, in the biological sample, the individual being assessed is at greater risk than if the FDP marker level is less than about 1.0 μg/ml; the CRP level is less than 3.0 mg/L and HSP70 is not detectable.

The methods can be performed to assess various subjects, typically patients. In some embodiments, the subject is a mammal, e.g., a human. The subject can be, for example, a patient known to have CAD (i.e., with confirmed CAD), such as significant or insignificant CAD, stable CAD, or one suspected of having CAD. In some aspects, the subject is a patient known to have CAD, a patient with significant CAD or a patient with insignificant CAD. In some cases, the subject is a patient suspected of having CAD. In some aspects, the subject is one who is not presenting with, has not presented with, or has no symptoms of CAD or with symptoms of other cardiovascular outcomes. In one aspect, the subject is one who has had a recent acute coronary syndrome (ACS). In another aspect, the subject is a patient with or without known CAD or with or without symptoms of CAD but who has been found to have a high coronary calcium score, or been tested in an FRS, coronary calcium test, or second-tier blood test indicating the subject is at an intermediate or high-risk. In another aspect, the subject is a patient with significant CAD who has not had an AMI event within the last 30 days.

In some embodiments, the methods further include comparing the level of one or more, typically each, of the plurality of biomarkers measured in the test biological sample to a control level of the respective biomarker. In one aspect, the comparison involves measuring the level of one or more, e.g., each, of the plurality of biomarkers in a control sample. Typically, the control level so measured is then compared with the level measured in the test biological sample. Alternatively, the methods can include simply comparing a previously determined or predefined control level to the level measured in the test biological sample. For example, the control level of each biomarker can be calculated from data, such as data including the levels of the biomarker in control biological samples from a plurality of control subjects. Often, the control subjects and the subject under assessment are of the same species. In one example, the test biological sample and the control samples comprise plasma or serum. In one aspect, the risk of the adverse cardiovascular outcome in the subject is increased if the levels in the test biological sample are higher than the control levels.

In some examples, if the levels of the at least two biomarkers are significantly different (or higher) than their respective control levels, then the subject, e.g., the mammal, is assessed or identified as at increased probability of CAD or myocardial infarction or death.

The samples, e.g., the test biological samples and control samples, can be any biological sample. Typically, the sample is a body fluid or tissue obtained from the subject. Among the sample types for use with the methods are whole blood, blood fractions, blood components, plasma, platelets, serum, cerebrospinal fluid (CSF), bone marrow, urine, tears, milk, lymph fluid, organ tissue, nervous system tissue, non-nervous system tissue, muscle tissue, biopsy, necropsy, fat biopsy, fat tissue, cells, feces, placenta, spleen tissue, lymph tissue, pancreatic tissue, bronchoalveolar lavage (BAL), and synovial fluid. In some embodiments, the test biological sample and/or the control sample is whole blood, blood fractions, blood components, plasma, platelets, serum, or urine.

Detection or measurement of the biomarker levels can be carried out using any of a number of known methods. In one aspect, the measuring or detecting is carried out by immunoassay, for example, by combining the biological sample being assessed or sample derived therefrom with antibodies that specifically bind to the plurality of biomarkers, under conditions under which binding of the antibodies with their respective partners occurs, such as by ELISA. In one aspect, the levels of the FDP marker (e.g., mixture of FDPs) are measured using the DR70® assay. In another example they include one or more FDPs detected by a Fibrinogen ELISA assay that uses either anti-Fibrinogen polyclonal or multiple monoclonal antibodies. In another aspect, the CRP gene product (e.g., CRP protein) is measured using a high-sensitivity CRP (hs-CRP) assay.

Also provided are methods of treatment, for example, of subjects assessed by the above-described methods. In some embodiments, the methods are carried out by assessing or determining the risk of an adverse cardiovascular outcome or detecting levels of a plurality of biomarkers in a subject by any of the above-described methods and treating, altering or modifying treatment of, or discontinuing treatment of the subject, for example, for the adverse cardiovascular outcome. In some cases, the methods further include a step of first treating the subject for a cardiovascular event or outcome, prior to determining the risk.

In some aspects, such methods are performed in an iterative fashion. For example, in some cases, the methods further include repeating the assessment or determination step following treatment, such as after a certain period of time following treatment, for example, 1, 2, 3, 4, 5, 6, 7, 8, 9, 10 11, or 12 months or more following treatment, for example, to determine whether risk and/or disease has been favorably altered by treatment, and/or to determine whether additional, e.g., more aggressive, treatment is needed. For example, a determination after such repetition that the biomarker levels have not decreased or have not substantially decreased, can indicate that risk has not been favorably altered and/or that additional, e.g., more aggressive treatment is necessary. Thus, the methods in some aspects further include then administering additional therapy to the subject. In some aspects, the treatment methods include determining whether a subject is at increased risk (has increased probability) of having adverse cardiovascular outcome, e.g., by the above-described methods, and selecting a particular treatment course if the subject has increased probability of having adverse cardiovascular outcome.

Also provided are systems, e.g., kits, for performing the diagnostic methods, such as kits containing one or more agents that specifically bind to or hybridize to the biomarkers, typically an agent that specifically binds or hybridizes to each of the plurality of biomarkers, in any combination as described above.

### BRIEF DESCRIPTION OF THE DRAWINGS

FIG. 1 shows a summary of baseline demographics, by biomarker, for patients in the study described in Example 2A.

FIG. 2 shows risk scores (hazard ratios) for future risk of death or MI using the presence of 1, 2, or 3 elevated biomarkers in patients with significant CAD, as described in Example 2A (where the FDP marker was deemed elevated if greater than 1.0 μg/ml; the level of CRP was deemed elevated if greater than about 3.0 mg/L and HSP70 was deemed elevated if detectable). Numbers shown in front of parentheses represent median HR; numbers in parentheses represent confidence intervals.

FIG. 3 shows risk scores (hazard ratios) for future risk of death or MI using the presence of 1, 2, or 3 elevated biomarkers in patients with insignificant CAD as described in Example 2A (where the FDP marker was deemed elevated if greater than 1.0 μg/ml; the level of CRP was deemed elevated if greater than about 3.0 mg/L and HSP70 was deemed elevated if detectable). Numbers shown in front of parentheses represent median HR; numbers in parentheses represent confidence intervals.

FIG. 4 shows 1-year event rates (percentage of a given group of patients having an event (either AMI or death) within 1-year), for patients with significant CAD (panel A) and insignificant CAD (panel B) in the following groups: 0 biomarkers elevated, 1 biomarker elevated, 2 biomarkers elevated, and 3 biomarkers elevated, as determined in Example 2A (where the FDP marker was deemed elevated if greater than 1.0 μg/ml; the level of CRP was deemed elevated if greater than about 3.0 mg/L and HSP70 was deemed elevated if detectable). Y-axis shows percentage of patient group with an event within one year. In each graph, the four bars, from left to right, represent: (1) patients with 0 biomarkers elevated (40% of patients), (2) patients with 1 biomarker elevated (40% of patients), (3) patients with 2 biomarkers elevated (15% of patients), and (4) patients with 3 biomarkers elevated (5% of patients).

FIG. 5 shows event-free survival curves for participants in the study described in Examples 2A and 2B with significant coronary artery disease (FIG. 5A) and those with insignificant coronary artery disease (FIG. 5B), grouped according to the number of biomarkers deemed elevated as described in Example 2A.

### DETAILED DESCRIPTION

Available CAD diagnostic and predictive methods have not been entirely effective, for example, in identifying the majority of individuals in who are at risk of acute myocardial infarction (AMI) or death. Cardiac markers serve an important role in the early detection and monitoring of cardiovascular disease. Markers of disease are typically substances found in a bodily sample that can be easily measured. The measured amount can correlate with underlying disease pathophysiology, presence or absence of a current or imminent cardiac event, or probability of a cardiac event in the future. Current markers, even in combination with other measurements or risk factors, do not adequately identify patients at risk of CAD and its adverse effects.

The invention relates in part to the discovery that the levels of a combination of biomarkers associated with inflammation, thrombotic disease, cellular stress, and autoimmune diseases (and in particular, inflammation, thrombotic disease, and cellular stress) and optionally infection, can be measured and the levels in the aggregate used in assessing the risk of adverse cardiac events in humans. Inflammation, cellular stress, infection, and thrombotic and autoimmune diseases can contribute, through individual pathways, to an aggregate burden of risk for CAD and its adverse consequences, particularly for plaque rupture (also known as acute myocardial infarction (AMI)) and death. Activation of diverse pathophysiologic processes that include inflammatory, cellular stress, infectious, thrombotic, and immune pathways, contribute to the development of CAD, subsequent plaque rupture and adverse outcomes. In some embodiments, provided are methods involving the measurement of biomarkers, such as CRP gene products and FDP marker (including two or more FDPs), in some cases in combination with HSP70 and/or CMV to perform risk assessment of adverse cardiac outcome in a subject. Such methods are useful, for example, to identify subjects presenting with known CAD and for whom the probability of undergoing a significant adverse cardiovascular outcome, such as a plaque rupture is elevated. The assay is also useful to identify subjects (in identifying individuals) who do not have a prior diagnosis of CAD, who have CAD but in which stenoses are deemed not hemodynamically significant, and/or who are at an increased risk of an adverse cardiac outcome.

### DEFINITIONS

In order to facilitate an understanding of the provided embodiments, selected terms used in the application will be discussed below.

The term “adverse cardiovascular outcome” emanating from atherosclerosis refers to coronary artery disease (CAD), ischemic heart disease (IHD), angina pectoris, AMI, death, stroke and peripheral vascular disease.

The term “coronary artery disease” refers to atherosclerotic disease of the coronary arteries, but also infers probable atherosclerotic disease of the peripheral arteries such as those supplying the legs and the brain, and includes the consequences of CAD, such as myocardial infarction and death, angina pectoris, stroke and peripheral vascular disease.

The term “biomarker” refers to a distinctive biological or biologically derived indicator of a process, event, or condition. Biomarkers as used herein encompass, without limitation, gene products, including proteins, nucleic acids, and metabolites, together with their polymorphisms, mutations, variants, modifications, subunits, fragments, protein-ligand complexes, and degradation products, protein-ligand complexes, elements, related metabolites, and other analytes or sample-derived measures that are associated with a biological state. Biomarkers can also include mutated proteins or mutated nucleic acids. Biomarkers preferably include inflammatory, infection, thrombotic or autoimmune biomarkers.

The term “inflammation biomarker” or “biomarker of inflammation,” as used herein, refers to a biomarker that is an indicator of inflammation. Exemplary inflammation biomarkers include C-reactive protein (CRP), interleukin (IL)-6, and serum amyloid A protein, homocysteine.

The term “biomarker of infection” or “infection biomarker,” as used herein, refers to a biomarker that is an indicator of infectious diseases. A list of potential infection biomarkers (biomarkers of infection) can be found at the Infectious Disease Biomarker Database (IDBD http://biomarker.cdc.go.kr and http://biomarker.korea.ac.kr). Exemplary infection biomarkers include gene products of (including proteins): CRP, anti-cytomegalovirus (CMV) antibody, Chlamydia pneumonia, herpes simplex virus (HSV) types 1 and 2, Helicobacter pylori, and hepatitis A virus, as well as periodontal pathogens.

“Autoimmune disease biomarker,” or “biomarker of autoimmune disease,” as used herein, refers to a biomarker that is an indicator of autoimmune disease. Three groups of gene products, e.g., proteins, are reflective of the autoimmune disease process: (1) degradation products arising from destruction of affected tissues, (2) enzymes that play a role in tissue degradation and (3) cytokines and other proteins associated with immune activation (Prince, H. E., Biomarkers, 2005, Nov. 10, Suppl 1: S44-49). Exemplary autoimmune disease biomarkers include gene products of (including proteins): antibody to Heat Shock Protein 60 (anti-HSP60), Heat Shock Protein 70 (HSP70), aggrecan fragments, C-propeptide of type II collagen and cartilage oligomeric matrix protein, matrix metalloprotease (MMP)-1, MMP-3 and MMP-1/inhibitor complexes thioredoxin, IL-16 and tumour necrosis factor (TNF)-alpha, neurofilament light protein and glial fibrillary acidic protein, MMP-2 and MMP-9 and TNF-alpha and soluble vascular adhesion molecule-1.

“Cellular stress biomarker,” or “biomarker of cellular stress,” as used herein, refers to a biomarker, the levels of which increase when a cell is exposed to stress. Exemplary cellular stress biomarkers include Heat Shock Protein (HSP) 70 (HSP70), and certain other HSPs, such as HSP32, HSP27, HSP72, HSP90, HSP47, as well as ubiquitin, and Hsc70, and cellular stress biomarkers discussed by Rajdev and Sharp, Toxicologic Pathology, 28(1) 105-112 (2000); Zhou et al., Circulation, 110: 207-213 (2004).

The term “biomarker of thrombosis,” or “thrombosis biomarker,” as used herein, refers to a biomarker that is an indicator of thrombosis. Some examples include fibrinogen, prothrombin 1.2, tissue plasminogen activator antigen (tPA), plasminogen activator inhibitor-1 (PAI-1), and FDP markers, such as an FDP marker that includes a mixture of at least two fibrin and fibrinogen degredation products (FDPs), such as two or more of fragments X, Y, D, D-dimer, and E fragment Y, and initial plasmin digest products (IPDP).

As used herein, the terms “fibrin degradation product(s),” “fibrin and fibrinogen degradation product(s),” and “FDP” refer to one or more fragments produced when either fibrin or fibrinogen is degraded. FDPs include four principal FDPs, called X, Y, D, and E fragments (fragment X, fragment Y, fragment D, and fragment E) that are liberated in various combinations. Cleavage of fibrinogen by plasmin produces fragments D and E as the primary end-products. Thrombin converts fibrinogen to fibrin. When a fibrin clot is broken down by plasmin, the last fragment to be degraded (containing two D and one E subunits) is split, releasing the E fragment and also two D fragments covalently linked together (called “D-dimer”). This D-dimer is produced from fibrin, not fibrinogen degradation. Accordingly, the FDP biomarker can include the presence of one or more of X, D, and E fragments. In one example, the FDP biomarker includes fragment Y; in one example, it includes one or two distinguishable forms of initial plasmin digest product (IPDP). In some embodiment, the provided methods and systems detect a the level of a FDP marker; in one aspect, this FDP marker includes a mixture of at least two FDPs, such as fragments D, E, and D-dimer, and in some aspects further including one or more of fragments X, Y, and IPDP. In one example, the FDP marker includes one, more, or all FDPs detected by the DR-70® ELISA assay. In another example, the FDPs include one or more FDPs described in International Application Publication Number WO 2010/114514 A1. In another example they include one or more FDPs detected by a Fibrinogen ELISA assay using either anti-Fibrinogen polyclonal or multiple monoclonal antibodies.

“C-reactive protein” is also known as “hsCRP”, or “CRP,” and is a marker of the reactant plasma protein component of the inflammatory response. CRP is a protein produced by hepatocytes as part of the non-specific acute phase response to inflammatory conditions. It is used to diagnose and monitor a wide variety of infectious diseases.

“Heat shock protein 70” is also known as“HSP70” “HSP73” “HSPA8” Other family members include HSP 70-1, HSP 70-2, HSP 70-4, HSP 70-4L, HSP 70-5, HSP 70-6, HSP 70-7, HSP 70-8, HSP 70-9, HSP 70-12a, HSP 70-14. Increased levels are found during conditions in which cells are exposed to stress. Thus, HSP70 is among the cellular stress biomarkers.

“Cytomegalovirus” “CMV” is a part of the herpes family of viruses. Other family members include herpes simplex virus type 1 (HSV-1 or HHV-1) and herpes simplex virus type 2 (HSV-2 or HHV-2), varicella zoster virus (VZV), human herpesvirus (HHV)-6, HHV-7, and HHV-8. The biomarker detected for CMV includes CMV antibody (CMV-Ab).

The term “mammal” or “subject” refers to such organisms as mice, rats, rabbits, goats, horse, sheep, cattle, cats, dogs, pigs, more preferably monkeys and apes, and most preferably humans. In some embodiments, the subject is a human, and the test or biological sample, which can be a test sample or a control sample, used is a bodily fluid or bodily tissue.

The term “bodily fluids” as used herein include circulating and non-circulating fluids. Examples of circulating fluids include blood, CSF, and lymph fluid. Examples of non-circulating fluids include synovial fluid.

The bodily fluid is selected from the group consisting of whole blood, blood fractions, blood components, plasma, platelets, lymph fluid, saliva, serum, gastric juices, bile, cerebrospinal fluid (CSF), bone marrow, tears, milk, organ tissue, nervous system tissue, non-nervous system tissue, muscle tissue, biopsy, necropsy, fat biopsy, fat tissue, cells, feces, placenta, spleen tissue, lymph tissue, pancreatic tissue, bronchoalveolar lavage (BAL), synovial fluid and urine. Most the body fluid is blood.

Biomarkers in a biological sample can be measured by a variety of methods, including, but not limited to, a method selected from the group consisting of chromatography, immunoassay, enzymatic assay, and spectroscopy; the biomarker can be directly or indirectly detected.

“Marker level” means the amount of the marker in the sample, and refers to units of concentration, mass, moles, volume, concentration, or other measure indicating the amount of marker present in the sample.

The chromatographic method used can be high performance liquid chromatography (HPLC) or gas chromatography (GC). The spectroscopic method used can be, for example, ultraviolet spectroscopy (UV or UV/Vis spectroscopy), infrared spectroscopy (IR), or nuclear magnetic resonance spectroscopy (NMR).

The immunoassay preferably detects a (at least one; one or more) biological marker selected from the group consisting of CRP, FDP, HSP70 and anti-CMV Ab. Preferably, the immunoassay detects the biomarker in the test sample using anti-marker antibodies or other immunologically active molecules. The immunoassay can be an enzyme-linked immunosorbant assay (ELISA). FDP can be measured using the ELISA referred to as DR70. FDP can also be measured by a Fibrinogen ELISA using either anti-Fibrinogen polyclonal or multiple monoclonal antibodies.

The term “control level of a biomarker” refers to the levels of biomarker in a population of normal subjects. The subject under assessment and the control subjects are of the same species (e.g., a human subject is being assessed and the control subjects are also humans).

As used herein, “significant” coronary artery disease (CAD) refers to CAD having one or more lesions with greater than or equal to (≧) 150% coronary stenosis, as determined angiographically. Thus, “insignificant” or “non-significant” CAD refers to CAD with no coronary stenosis greater than or equal to (≧) 150% (i.e., only coronary stenosis of less than (<) 50%), as determined angiographically.

### EMBODIMENTS

Provided are diagnostic, prognostic, and predictive methods and systems (e.g., kits) for determining risk of adverse cardiovascular outcomes in subjects, such as humans. In one embodiment, the methods and systems detect or predict coronary artery disease (CAD) or related disease, or the risk of developing CAD or related disease, in the absence of known disease in mammals. In another embodiment, the methods and systems detect or predict an adverse effect of CAD, such as myocardial infarction (MI), e.g., acute MI (AMI) or death, such as within a given period of time, such as a year following performance of the methods, such as methods for assessing increased probability of AMI. Thus, the methods may be performed on a subject known to have or suspected of having CAD, e.g., significant or insignificant CAD, a subject not presenting with any symptoms of CAD, having stable CAD, or one having had a recent acute coronary syndrome (ACS), including one who has not had a MI event within a recent time period, such as within 30 days. In some aspects, the methods are useful in identifying subjects presenting with known CAD and for whom the probability of undergoing a significant adverse cardiovascular outcome, such as a plaque rupture, is elevated. The assay also has utility in identifying subjects from the general population without a prior diagnosis of CAD who are at an increased risk of an adverse cardiac outcome, or for assessing increased risk (probability) of myocardial infarction, death, or stroke in a mammal.

The methods generally involve detecting or measuring the presence, absence, or levels of a plurality of biomarkers in the subject, generally in a test biological sample obtained from the subject. In some aspects, the levels of the plurality of biomarkers, for example, in the aggregate, indicate a risk of an adverse cardiovascular outcome, such as those described above, in the subject.

In one aspect, the methods include a step of providing or obtaining the biological sample for use in the methods. The sample can be any biological sample type, such as from a cell, tissue, or body fluid, and generally is a sample derived from blood, such as whole blood, blood fractions, blood components, plasma, platelets, or serum. Other exemplary sample types include cerebrospinal fluid (CSF), bone marrow, urine, tears, milk, lymph fluid, organ tissue, nervous system tissue, non-nervous system tissue, muscle tissue, biopsy, necropsy, fat biopsy, fat tissue, cells, feces, placenta, spleen tissue, lymph tissue, pancreatic tissue, bronchoalveolar lavage (BAL), and synovial fluid.

The plurality of biomarkers includes at least two biomarkers and generally includes one or more thrombosis biomarkers, which generally includes an FDP marker. As described above, the FDP marker typically is a mixture of at least two FDPs, such as fragments D, E, and D-dimer, and in some aspects further including one or more of fragments X, Y, and IPDP, such as one, more, or all FDPs detected by the DR-70® ELISA assay or FDPs described in International Application Publication Number WO 2010/114514 A1. In another example FDP can be measured by a Fibrinogen ELISA that uses anti-Fibrinogen polyclonal or multiple monoclonal antibodies. The panel of biomarkers generally also includes at least one inflammatory (inflammation) biomarker, at least one autoimmune disease or cellular stress biomarker, and in some cases an infection biomarker, or a combination thereof. Exemplary inflammation biomarkers include C-reactive protein (CRP) gene products, such as CRP protein. Exemplary cellular stress biomarkers include Heat Shock Protein 70 (HSP70) gene products, such as HSP70 proteins. In some embodiments, the panel includes a thrombosis biomarker, an inflammation biomarker, and an autoimmune disease or cellular stress biomarker, and in some cases, a thrombosis biomarker, an inflammation biomarker, and a cellular stress marker; in some aspects, it further includes an infection biomarker.

Thus, in particular embodiments, the method comprises measuring the biomarkers CRP and an FDP marker; measuring CRP, FDP marker and HSP70; CRP, FDP marker, and anti-CMV Ab or measuring CRP, FDP marker, HSP70 and anti-CMV Ab to determine risk assessment of adverse cardiac outcome in a subject.

In one aspect, the methods include the steps of measuring a level of at least two biomarkers (HSP70, CRP, FDP and/or anti-CMVAb) in the sample from the mammal (referred to as a test biological sample); (b) comparing the level of the biomarkers measured in the test sample with control level for each; and (c) determining if the level of the two or more biomarkers are significantly different that that of each control biomarker level, wherein if the level of each of the two or more biomarkers is significantly greater than their respective control levels, the individual is at increased risk.

In one embodiment, the method measures levels of CRP and an FDP marker. In another embodiment, the method measures levels of CRP, an FDP marker and HSP70. In an alternative embodiment, the method measures levels of CRP, an FDP marker and anti-CMV Ab. In an alternative embodiment, the method measures levels of CRP, an FDP marker, HSP 70 and anti-CMV Ab.

In another embodiment, the method measures the level of HSP70, C-reactive protein (CRP), an FDP marker, and/cytomegalovirus (CMV) antibody by providing a bodily fluid; contacting the homogenate with an biomarker-specific binding reagent under conditions that allow binding of the reagent to HSP70, C-reactive protein (CRP), an FDP marker, and/or anti-cytomegalovirus antibodies, if present, to form a complex; and detecting HSP70, CRP and an FDP marker and anti-cytomegalovirus antibodies, if any, in said subject biological sample by its immunoassay.

In some examples, the risk of the given adverse outcome is deemed increased, such as by 2, 3, 4, 5, 6, or more times, if the levels are higher than control levels, or significantly higher, or a particular degree higher, e.g., deemed elevated (compared to a situation where the levels were not as such). In one example, the FDP marker is elevated if greater than 1 microgram per milliliter. In one example, the CRP gene product is elevated if greater than three milligrams per milliliter of sample. In one example, the HSP70 gene product is elevated if detected.

In some aspects, the methods include providing a homogenate of bodily tissue or other biological sample and contacting the homogenate or sample with an biomarker-specific binding reagent under conditions that allow binding of the reagent to the biomarker, if present, to form a complex. In some aspects, detecting the biomarkers, if any, in the biological sample by its immunoassay. In this embodiment, a reagent can be one that binds to one biomarker or multi-reagents, each of which binds to a different biomarker or can bind to more than one biomarker. Thus, the levels of biomarkers can be measured by an immunoassay. Preferably, the immunoassay detects the biomarker in the test sample using anti-marker antibodies. The immunoassay can preferably be an enzyme-linked immunosorbant assay (ELISA). In one embodiment, the ELISA for the FDP marker is DR70®. DR70® is a diagnostic cancer test cleared by the USFDA for monitoring colorectal cancer DR70® measures the FDP marker levels produced from multiple pathways, unlike other FDP assays which only measure one pathway or one pathway product. In other examples, FDP products can be measured using polyclonal anti-Fibrinogen antibodies and/or monoclonal anti-Fibrinogen antibodies, typically multiple monoclonal antibodies, such as by a Fibrinogen ELISA containing either polyclonal anti-Fibrinogen antibodies or multiple monoclonal anti-Fibrinogen antibodies.

In some embodiments, the CRP biomarker (e.g., CRP protein) is measured using a high-sensitivity CRP (hs-CRP) assay, such as the assays described by Roberts et al., Clinical Chemistry, 46:4, 461-68 (2000), those described by Shine B, et al., Clin Chim, Acta, 1981; 117: 13-23, the BNII automated system commercially available from Dade-Behring Inc., and/or rate nephelometry (Behring NA latex CRP; Behring, Germany) and/or the test available from GenWay Biotech., Inc., catalog number 40-052-115042.

In some embodiments, the levels of at least two biomarkers are compared with control levels of such biomarkers, such as the level of their respective predetermined values (control value). In preferred embodiments, the predetermined value is indicative of a normal cardiac condition.

This predetermined value can be determined using the known methods or methods described herein, and can be specific for a particular patient or generic for a given population. In one embodiment, the control biomarker level (control level for a biomarker) is a level (or a range of levels) for a biomarker (i.e., FDP marker, CRP, HSP70, anti-CMV Ab) in a population of healthy subjects (subjects who do not have CAD, such as subjects with <20% coronary stenosis).

The predetermined (control) value is preferably obtained from a human of approximately the same age as the subject being assessed. (from whom the biological sample, also referred to as the test sample) is obtained. Additional characteristics, such as gender, ethnicity, smoking history, weight/other condition (e.g., diabetes and cardiovascular risk factor) can also be taken into consideration. In certain embodiments, the predetermined value may have been established by prior measurement of the particular patient's marker levels, such as when the patient had cardiac symptoms (e.g., chest pain) without prior history and/or marker levels in patients with confirmed CAD.

In a further embodiment, the method is a method of treating a subject, comprising determining whether a subject has increased probability of having adverse cardiovascular outcome, and selecting a particular treatment course if the subject has increased probability of having adverse cardiovascular outcome. The prediction of an increased probability of an adverse cardiovascular outcome can be in a subject presenting symptoms of CAD, or in one who does not so present.

Also provided are systems, e.g., kits, for use in the methods, such as kits including a plurality of agents that specifically bind to and/or detect the plurality of biomarkers, in any combination as described above. Kits useful in the methods provide cost-effective and rapid tests that can be used to assess the probability of adverse cardiac outcome, among other cardiac conditions, acute myocardial infarction (AMI). The kits comprise at least one biomarker specific to one of the two or more biomarkers to be assessed in a biological sample. For example, a kit may comprise a specific biomarker that detects FRP (e.g., and a specific biomarker that detects CRP).

In addition, in certain embodiments, the provided diagnostic methods are useful for assessing the probability of having or of developing CAD before symptoms occur (in persons who do not present with CAD; persons not known to have CAD), or assessing risk of myocardial infarction or death in persons with known CAD, whether the severity of coronary stenoses is deemed to be significant or non-significant. In addition, the methods and compositions can be used to monitor the effectiveness of therapeutic interventions designed to relieve ischemia and heart failure. Some embodiments for monitoring include testing the subject during or after therapeutic intervention at intervals of 3 months, 6 months, or one year, as needed.

Also provided are methods of treatment, including performing the diagnostic or prognostic methods as described and subsequently treating, discontinuing treatment, or altering treatment, of the subject, e.g., based on the results of the assay. In some aspects, the methods further include first treating the subject.

In some embodiments, the methods include statistical analyses, including risk assessment algorithms. In one example, the provided methods and systems are able to discriminate between individuals who will or are more likely to have a given outcome, e.g., AMI or death, and those who will not or are more likely not to have such an outcome, for example, within a given time period.

In some aspects, using well-known statistical analysis methods, hazard ratios (HRs) are calculated, e.g., to represent the relationship between one or more given value(s) or covariate(s), such as a particular level of one or more biomarkers or that one or more biomarkers is deemed elevated or positive as described herein, and a particular outcome, endpoint, or event, such as the occurrence of death or AMI. Thus, the HR describes the risk of occurrence of the outcome, event, or endpoint associated with the value(s) or covariate(s).

In one aspect, the methods include use of a receiver operating characteristic (ROC) curve and the probabilistic interpretation of the area under the ROC curve (AUC; C-statistics). An ROC curve is a plot of the sensitivity of a given test or prediction model (plotted on the y-axis) versus the test or model's false-positive rate (FPR; 1 specificity) (plotted on the x-axis). Each point on the graph is generated by using a different cut-point. In some examples, the cut-point is the concentration or amount of a given biomarker, at or above which the biomarker is deemed “elevated” in the sample. The AUC (C-statistics) is the area under the plot for all possible cut-off values. Thus, the ROC curve allows direct visual comparison of two or more tests on a common set of scales at all possible cut-points. A test (e.g., a risk prediction model) with a C-statistics of 1.0 is perfectly accurate (because the sensitivity is 1.0 when the FPR is 0.0); a test with a C-statistics of 0.0 is perfectly inaccurate. Thus in some embodiments, utilizing ROC curves, C-statistics are generated and compared for risk prediction models, such as those using the biomarkers, when individual biomarkers are added, as well as when subsets of biomarkers are being evaluated. Overall, the closer the C-statistics is to 1.0, the better the risk prediction model.

The following examples are offered to illustrate but not to limit the invention.

### EXAMPLES

### Example 1

### Clinical Validation Study

This example demonstrates that determining an aggregate biomarker risk score, including levels of C-reactive protein (CRP), a fibrin and fibrinogen degradation product (FDP) marker (including a mixture of multiple FDPs), and Heat Shock Protein 70 (HSP70), can assess or indicate future risk of adverse events in subjects. Cytomegalovirus antibody (anti-CMV Ab) and antibody to Heat Shock Protein 60 (anti-HSP60) also were assessed. CRP was measured using a high-sensitivity CRP (hs-CRP) assay (GenWay Biotech., Inc., catalog number 40-052-115042). The FDP marker was measured using the DR-70® ELISA assay.

Methods:

1500 subjects were selected in a case-control study from the Emory Cardiology Biobank, a registry of patients undergoing cardiac catheterization; included those: (1) with angiographic CAD who suffered death, MI at 2 years of follow-up after enrollment (n=200 subjects); (2) with angiographic CAD without MI or death (n=800) of which 300 subjects underwent revascularization during 2 years of follow up after enrollment; and (3) with non-significant CAD based on angiography (n=500). Groups were matched for age, gender and cardiovascular risk factors using propensity scoring (Rosenbaum, P. R. and Rubin, D. B., Biometrika, 1983, 70(1), 41-55; http://en.wikipedia.org/wiki/Propensity_score_matching). Serum biomarkers were measured using ELISA (GenWay® Inc.). Cox proportional hazard survival analyses were performed with models further adjusted for age, BMI, sex, race, smoking, diabetes, and hypertension. Data were analyzed as continuous variables and with cut points assigned based on accepted values.

Results:

Levels of CMV antibody and anti-HSP60 were not significantly associated with any outcomes. For primary outcomes of death and MI at two years, with adjustments for standard cardiovascular risk factors, the association for each biomarker modeled continuously demonstrated a hazard ratio (HR) of 1.55 (p<0.0001). Modeling with cut points revealed hazard ratios of 1.89 (p<0.001) for CRP>3.0 mg/L; HR of 1.42 (p=0.005) for HSP70 when detectable; and HR of 2.04 (p<0.001) for FDP>1.0 μg/ml. HRs for the primary outcome using categorical risk scores calculated using all 3 significant biomarkers in aggregate revealed 1.68 (p=0.008) for 1 biomarker, 2.64 (p<0.0001) for 2 biomarkers, and 3.76 (p<0.0001) for 3 biomarkers elevated. These results provided the basis for a scoring system and for an aggregate score.

The results showed that the measurement, particularly in the aggregate, of levels of CRP, an FDP marker, and HSP70, is a strong predictor of future risk of death and MI in patients without known CAD.

TABLE 1
Associations of CRP, HSP70 and DR70 with MI + death events,
using CAD as “control” group

| | Adjustment 1* | Adjustment 2** |
|---|---|---|
| | HR(95% CI); p-value | HR (95% CI); p-value |
| Separate | | |
| Models: | | |
| CRP at 3.0 | 1.94 (1.49, 2.51); <0.0001 | 1.78 (1.37, 2.32); <0.0001 |
| CRP at 6.7 | 1.84 (1.42, 2.37); <0.0001 | 1.71 (1.32, 2.21); <0.0001 |
| HSP70 at 0 | 1.44 (1.13, 1.85); 0.004 | 1.47 (1.14, 1.90); 0.003 |
| DR70 at 1.0 | 1.99 (1.54, 2.58); <0.0001 | 1.93 (1.49, 2.51); <0.0001 |
| All in one | | |
| Model: | | |
| CRP at 3.0 | 1.75 (1.34, 2.28); <0.0001 | 1.59 (1.22, 2.09); 0.0007 |
| HSP70 at 0 | 1.25 (0.97, 1.61); 0.09 | 1.30 (1.00, 1.68); 0.05 |
| DR70 at 1.0 | 1.73 (1.33, 2.26); <0.0001 | 1.66 (1.27, 2.19); 0.0002 |
| CRP at 6.7 | 1.69 (1.30, 2.18); <0.0001 | 1.57 (1.22, 2.04); 0.0006 |
| HSP70 at 0 | 1.27 (0.99, 1.64); 0.06 | 1.31 (1.01, 1.70); 0.04 |
| DR70 at 1.0 | 1.78 (1.36, 2.32); <0.0001 | 1.72 (1.31, 2.25); <0.0001 |

*Adjusted for age, gender, race, BMI, ever smoking, HTN and diabetes
**Adding PCI history, previous MI, plavix and aspirin use to adjustment 1

TABLE 2
Comparison of Risk Score Models for Different Analysis Groups

| | All Participants* | | CAD group only as “control”** | |
|---|---|---|---|---|
| | All 3 markers, CRP at 3.0 | All 3 markers, CRP at 6.7 | All 3 markers, CRP at 3.0 | All 3 markers, CRP at 6.7 |
| | HR (95% CI); p-value | HR (95% CI); p-value | HR (95% CI); p-value | HR (95% CI); p-value |
| Unweighted continuous | 1.55 (1.37, 1.77); <0.0001 | 1.58 (1.39, 1.81); <0.0001 | 1.51 (1.33, 1.73); <0.0001 | 1.53 (1.34, 1.75); <0.0001 |
| Unweighted categorical | | | | |
| 1 vs 0 markers | 1.70 (1.16, 2.49); 0.006 | 1.80 (1.29, 2.50); 0.001 | 1.75 (1.19, 2.59); 0.005 | 1.87 (1.34, 2.61); 0.0002 |
| 2 vs 0 markers | 2.66 (1.81, 3.92); <0.0001 | 3.11 (2.19, 4.42); <0.0001 | 2.69 (1.81, 3.98); <0.0001 | 2.81 (1.96, 4.02); <0.0001 |
| 3 vs 0 markers | 3.79 (2.44, 5.89); <0.0001 | 3.40 (2.11, 5.47); <0.0001 | 3.50 (2.22, 5.51); <0.0001 | 3.27 (2.01, 5.33); <0.0001 |
| Weigited categorical (median) | 1.98 (1.50, 2.60); <0.0001 | 2.12 (1.65, 2.72); <0.0001 | 1.95 (1.47, 2.57); <0.0001 | 1.95 (1.52, 2.52); <0.0001 |
| Specification of Markers: | | | | |
| CRP only vs. 0 markers | 1.69 (1.09, 2.62); 0.02 | 2.09 (1.32, 3.31); 0.002 | 1.73 (1.10, 2.70); 0.02 | 2.02 (1.27, 3.22); 0.003 |
| HSP70 only vs. 0 markers | 1.57 (0.99, 2.49); 0.05 | 1.63 (1.13, 2.36); 0.01 | 1.65 (1.04, 2.62); 0.03 | 1.74 (1.20, 2.54); 0.004 |
| DR70 only vs, 0 markers | 2.09 (1.04, 4.19); 0.04 | 1.96 (1.12, 3.42); 0.02 | 2.28 (1.13, 4.59); 0.02 | 2.08 (1.18, 3.65); 0.01 |
| CRP/HSP70 vs. 0 markers | 2.29 (1.50, 3.52); 0.0001 | 2.45 (1.55, 3.89); 0.0001 | 2.41 (1.56, 3.72); <0.0001 | 2.28 (1.43, 3.61); 0.0005 |
| CRP/DR70 vs, 0 markers | 4.42 (2.64, 7.40); <0.0001 | 6.94 (4.04, 11.93); <0.0001 | 3.71 (2.20, 6.27); <0.0001 | 4.63 (2.66, 8.06); <0.0001 |
| HSP70/DR70 vs. 0 markers | 1.89 (0.98, 3.64); 0.06 | 2.70 (1.74, 4.19); <0.0001 | 2.17 (1.09, 4.30); 0.03 | 2.65 (1.68, 4.17); <0.0001 |
| All three markers vs, 0 markers | 3.68 (2.37, 5.72); <0.0001 | 3.30 (1.07, 5.37); <0.0001 | 3.43 (2.17, 5.42); <0.0001 | 3.21 (1.97, 5.23); <0.0001 |

*Models adjusted for age, sex, race, BMI, ever smoking, HTN, diabetes, Gensini score
**Models adjusted for age, sex, race, BMI, ever smoking, HTN, diabetes, PCI history, previous MI, Plavix use, aspirin use

### Example 2A

### Clinical Validation Study

An additional study was carried out, with four patient groups: Cohort A—angiographically confirmed significant CAD (≧50% coronary stenosis)—with MI or death; Cohort B—Angiographically confirmed significant CAD without MI or death; Cohort C—Angiographically confirmed insignificant CAD (<50% coronary stenosis as determined angiographically) with MI or death; and Cohort D—Angiographically confirmed insignificant CAD (<50% coronary stenosis as determined angiographically) without MI or death—, 3,800 subjects total. 1,500 subjects were used in an initial testing study; 2,300 subjects were used in a subsequent validation study.

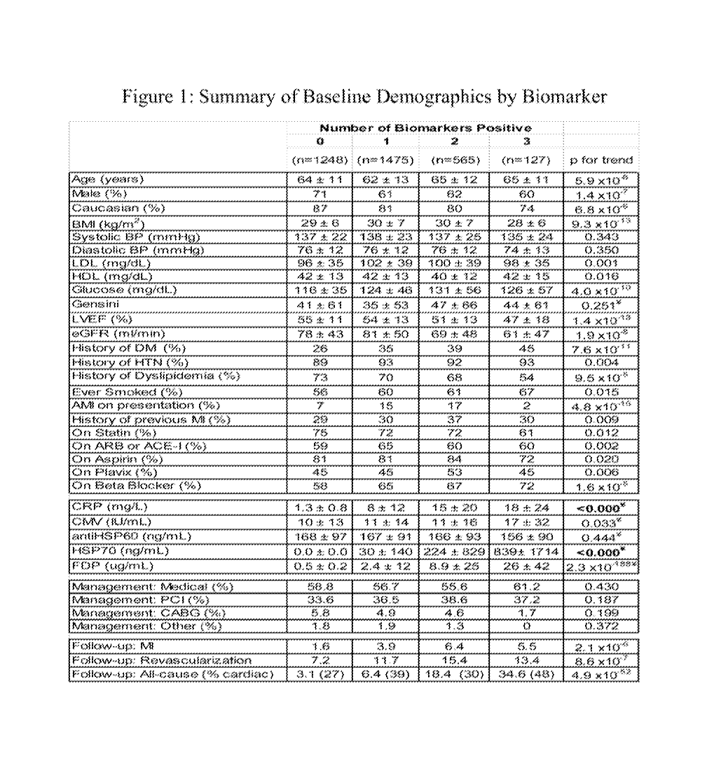

Blood was collected for sampling, all drawn prior to catheterization. Presence of severity and coronary artery disease was documented with angiograms. Data collection period was present, with mean follow-up of 2.75 years. The serum levels of CRP, HSP70 antigen, FDP marker (as described above), and CMV antibody were measured. CRP was measured using a high-sensitivity CRP (hs-CRP) assay, as described in (GenWay Biotech., Inc., catalog number 40-052-115042). Table 3, below, summarizes baseline demographics of the subjects by group. FIG. 1 shows baseline demographics according to number of biomarkers positive (i.e., “elevated,” as described below).

TABLE 3
Summary of Baseline Demographics by Group

| | Total | | CAD | |
|---|---|---|---|---|
| | Participants* | | participants only** | |
| | n = 2951 | | n = 1637 | |
| Age | 63.0 ± 11.4 | | 65.1 ± 10.6 | |
| Male Sex | 1869 | (63%) | 1149 | (70%) |
| African-American | 483 | (16%) | 219 | (13%) |
| Ever Smoker | 1721 | (58%) | 1045 | (64%) |
| BMI | 29.8 ± 6.5 | | 29.6 ± 6.0 | |
| Diabetes† | 954 | (32%) | 598 | (37%) |
| Hypertension‡ | 2679 | (91%) | 1551 | (95%) |
| Plavix Use | 1308 | (44%) | 977 | (60%) |
| Aspirin Use | 2372 | (80%) | 1456 | (89%) |
| Statin Use | 2099 | (71%) | 1330 | (81%) |
| Gensini Score, median | 11.0 | (0, 49.1) | 32.0 | (10.0, 91.0) |
| (Q1, Q3) | | | | |
| Previous MI | 866 | (30%) | 797 | (49%) |
| PCI History | 1181 | (40%) | 1061 | (65%) |
| CRP, mg/L (median (Q1, Q3) | 2.7 | (1.2, 6.6) | 2.5 | (1.0, 6.5) |
| HSP70, ng/mL (median | 0 | (0, 0) | 0 | (0, 0) |
| (Q1, Q3) | | | | |
| FDP, ug/mL (median | 0.54 | (0.36, 0.81) | 0.54 | (0.36, 0.82) |
| (Q1, Q3) | | | | |
| Number of Elevated | | | | |
| Biomarkers | | | | |
| 0 | 1141 | (39%) | 659 | (40%) |
| 1 | 1234 | (42%) | 651 | (40%) |
| 2 | 455 | (15%) | 266 | (16%) |
| 3 | 121 | (4%) | 61 | (4%) |

**For CAD participants only, after excluding acute MI and transplants, and missing on previous MI, PCI history or biomarker risk score, there were 1637 participants in the analysis; 190 of these participants had had death or MI events.
*For total participants, after excluding acute MI and transplants, and missing on previous MI, PCI history or biomarker risk score, there were 2951 participants in the analysis; 296 of these participants had had death or MI events.
†Diabetes defined as medication use or glucose >=200 mg/dL
‡Hypertension defined as medication use, SBP >140 or DBP >90
⋄CRP >3.0 mg/L, HSP70 >0 ng/mL, FDP >1.0 ug/mL

Cox proportional hazard survival analyses were performed with models further adjusted for age, sex, race, smoking, diabetes, and hypertension. CRP was considered “elevated” if greater than 3.0 mg/mL. HSP70 was considered “elevated” when detectable. The FDP marker was considered “elevated” when greater than 1.0 microgram/mL.

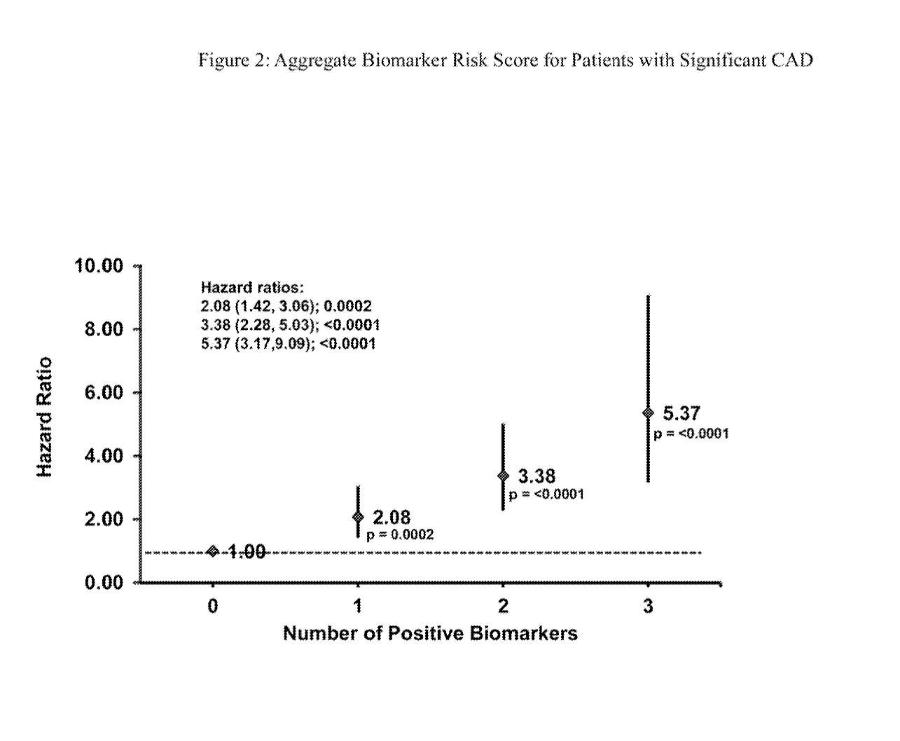

As shown in FIG. 2, in patients with significant CAD, the hazard ratios for future risk of death or MI using categorical risk scores calculated using various numbers of biomarkers in aggregate were 2.08 for 1 biomarker elevated, 3.38 for 2 biomarkers elevated, and 5.37 for 3 biomarkers elevated. Numbers in parentheses indicate HR (hazard ratio) confidence intervals.

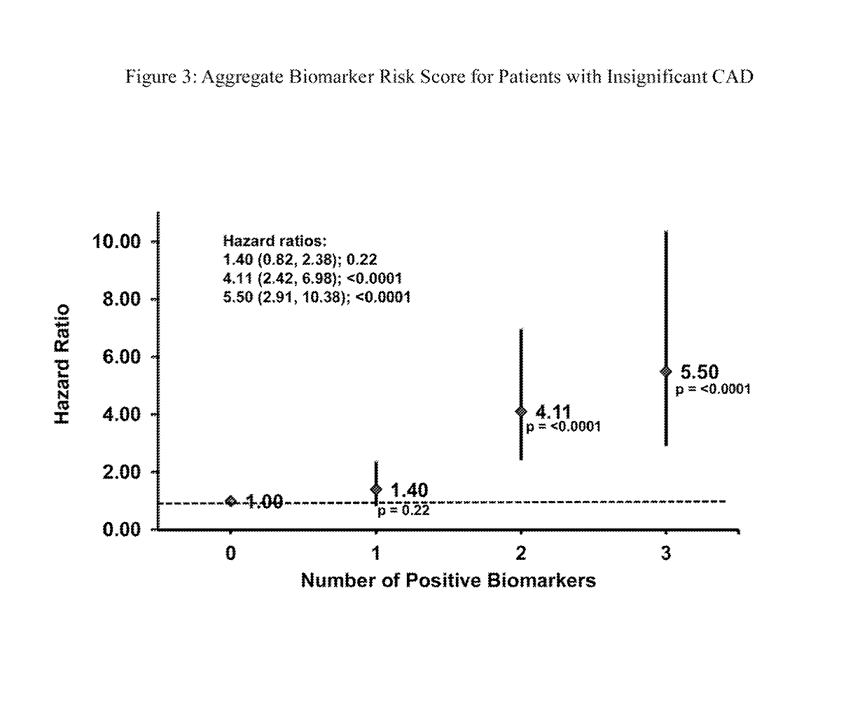

As shown in FIG. 3, in patients with insignificant CAD, the hazard ratios for future risk of death or MI using categorical risk scores calculated using various numbers of biomarkers in aggregate were 1.40 for 1 biomarker elevated, 4.11 for 2 biomarkers elevated, and 5.50 for 3 biomarkers elevated. Numbers in parentheses indicate HR (hazard ratio) for confidence intervals.

A detailed summary of results is presented in Table 4, below, with numbers listed according to median and confidence intervals in parentheses.

TABLE 4
Summary of Results of Study (Example 2)

| | | Significant CAD | Insignificant CAD |
|---|---|---|---|
| | All Participants* | participants only** | participants only^{†} |
| | HR(95% CI); p-value | HR(95% CI); p-value | HR(95% CI); p-value |
| All Biomarkers in Same | | | |
| Model | | | |
| CRP at 3.0 mg/L | 1.64 (1.31, 2.07); | 1.60 (1.20, 2.12); | 2.08 (1.40, 3.08); |
| | <0.0001 | 0.0011 | 0.0003 |
| HSP70 at 0 ng/mL | 2.39 (1.87, 3.04); | 2.37 (1.75, 3.22); | 1.82 (1.25, 2.65); |
| | <0.0001 | <0.0001 | 0.0018 |
| FDP at 1.0 ug/mL | 1.69 (1.34, 2.12); | 1.53 (1.15, 2.04); | 2.02 (1.38, 2.96); |
| | <0.0001 | 0.0040 | 0.0003 |
| Continuous Biomarker | 1.84 (1.64, 2.07); | 1.74 (1.50, 2.02); | 1.95 (1.61, 2.36); |
| Risk Score | <0.0001 | <0.0001 | <0.0001 |
| Categorical Biomarker | | | |
| Risk Score | | | |
| 1 vs 0 markers | 1.85 (1.35, 2.54); | 2.08 (1.42, 3.06); | 1.40 (0.82, 2.38); 0.22 |
| | 0.0001 | 0.0002 | |
| 2 vs 0 markers | 3.73 (2.71, 5.13); | 3.38 (2.28, 5.03); | 4.11 (2.42, 6.98); |
| | <0.0001 | <0.0001 | <0.0001 |
| 3 vs 0 markers | 5.66 (3.78, 8.49); | 5.37 (3.17, 9.09); | 5.50 (2.91, 10.38); |
| | <0.0001 | <0.0001 | <0.0001 |

*Adjustment for age, race, gender, BMI, ever smoking, HTN, diabetes, aspirin use, statin use, acute MI, previous MI, gensini score, hyperlipidemia and eGFR
**Adjustment for age, race, gender, BMI, ever smoking, HTN, diabetes, aspirin use, plavix use, statin use, PCI history, acute MI, previous MI, gensini score, hyperlipidemia, and eGFR
^{†}Adjustment for age, race, gender, BMI, ever smoking, HTN, diabetes, aspirin use, statin use, gensini score, hyperlipidemia, and eGFR
NOTE:
THESE MODELS ADJUSTED FOR AMI BUT STILL REMOVED TRANSPLANT

Using ROC curves, C-statistics were generated and compared for risk prediction models using the biomarkers versus traditional risk factors (base model). As shown in Table 5, below, addition of the three biomarkers (FDP marker, HSP70, and CRP) to traditional risk factors improved the C-statistic, demonstrating the utility of methods provided herein for assessing risk of cardiovascular outcomes as compared with available risk assessment tools.

TABLE 5
C-statistic for All Participant Model

| | C-statistic | |
|---|---|---|
| | Lower Bound, | |
| | Upper Bound) | p-value |
| Base Model** | 0.695 (0.665, 0.725) | — |
| +CRP, FDP, HSP 70 | 0.750 (0.723, 0.776) | 7.1311 × 10⁻⁸ |
| in One Model^{†} | | |

*Models adjusted for Age, Sex, Race, Diabetes, Hypertension, Smoking, BMI, PCI composite, Aspirin Use, Plavix Use, Statin Use, Old Micomposite, Previous AMI, Gensini Score, Lipid Composite, and eGFRCKD.
^{†}CRP considered elevated above 3.0 mg/mL, HSP 70 considered elevated when detected (cut at zero), FDP considered elevated above 1.0 microgram per mL were added to the baseline model.

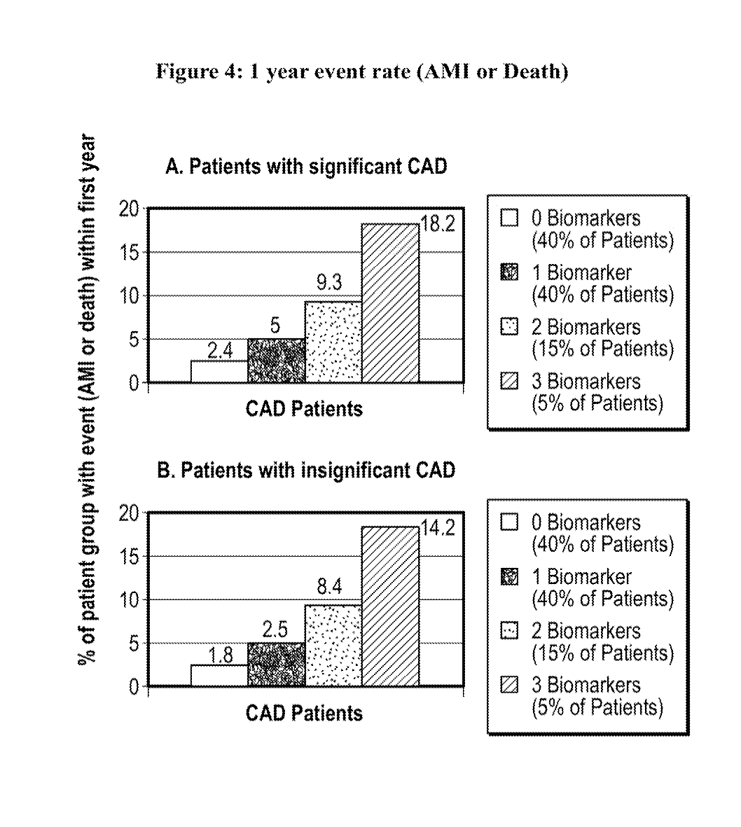

For patients with significant CAD and patients with insignificant CAD, the percentage patients in each of four groups (those in which 0, 1, 2, or all 3 biomarkers were considered elevated) having a major event (AMI or death) on an annual basis was calculated. The results, shown in FIG. 4, indicated that 18.2% of significant CAD patients in which all three biomarkers were elevated had a major event annually, compared to only 2.4% of CAD patients with normal biomarker levels, and that 14% of insignificant CAD patients in which all three biomarkers were elevated had a major event annually, compared to only 2.4% of CAD patients with normal biomarker levels.

### Example 2B

### Event-Free Survival Curves for Clinical Validation Study

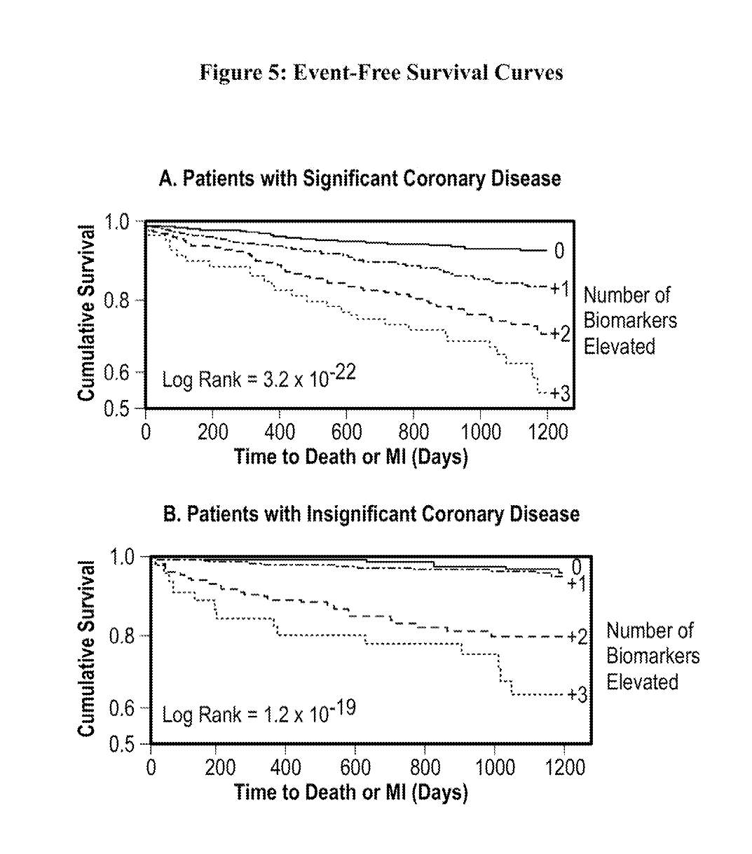

Event-free survival curves, shown in FIG. 5, were generated for the study described in Example 2A, to compare event-free survival (absence of AMI or death endpoints) for individuals with 0, 1, 2, and 3 of the biomarkers (CRP, HSP70, and FDP marker) elevated. For individuals with significant CAD (≧50% stenosis), only about two (2) % of those with none of the biomarkers elevated had an event (AMI or death) within 1,200 days of follow-up. In contrast, forty-five (45) % of individuals with significant CAD and all 3 biomarkers elevated had an event within 1,200 days of follow-up.

The results were similar for individuals with non-significant (insignificant) CAD. In this cohort, the risk of an event within 1,200 days was low for patients with zero or one (1) biomarker elevated. The presence of two (2) biomarkers elevated indicated a moderate risk of an event within 1,200 days (20% of participants within this cohort having 2 biomarkers elevated had such an event within 1,200 days post blood draw (20% event rate)). The presence of all three (3) of the biomarkers elevated indicated a high risk of an event within 1,200 days post blood draw. Patients with all three biomarker elevated represented 5% of the cohort with insignificant CAD. 35% of this group had an event within 1,200 days post blood draw (35% event rate).

The results demonstrate that in this study, neither percentage of stenosis (as indicated by comparing FIGS. 5A and 5B), nor elevation of a single biomarker, was a good predictor of risk of an adverse event. In contrast, elevated levels of two or three of the biomarkers, in the aggregate, predicted risk of an event (AMI or death).

1. A method of determining risk of an adverse cardiovascular outcome in a subject, the method comprising: measuring the level of each of a plurality of biomarkers in a test biological sample obtained from the subject, wherein:
the plurality of biomarkers comprises (i) a fibrin and fibrinogen degradation product (FDP) marker, wherein the FDP marker includes a mixture of at least two fibrin and fibrinogen degradation products (FDPs) selected from the group consisting of fragment D, fragment E, D-dimer, fragment X, fragment Y, and an initial plasmin digest product (IPDP), and (ii) at least one inflammation biomarker, autoimmune disease biomarker, or cellular stress biomarker; and
the levels of the plurality of biomarkers indicate a risk of an adverse cardiovascular outcome in the subject.

2. A method of determining risk of an adverse cardiovascular outcome in a subject, the method comprising: contacting a test biological sample from the subject with a panel of agents that specifically bind to a plurality of biomarkers, thereby measuring levels of the plurality of biomarkers, wherein:
the plurality of biomarkers includes a fibrin and fibrinogen degradation product (FDP) marker and at least one inflammation biomarker, autoimmune disease biomarker, or cellular stress biomarker;
the panel of agents includes an agent or agents that specifically binds or bind to at least two fibrin and fibrinogen degradation products (FDPs) selected from the group consisting of fragment D, fragment E, D-dimer, fragment X, fragment Y, and an initial plasmin digest product (IPDP); and
the levels so measured indicate a risk of an adverse cardiovascular outcome in the subject.

3-5. (canceled)

6. The method of claim 1, wherein the plurality of biomarkers includes the at least one inflammation biomarker, which comprises a C-reactive protein (CRP) gene product, and/or the at least one cellular stress biomarker, which comprises a Heat Shock Protein 70 (HSP70) gene product.

7. (canceled)

8. The method of any claim 1, wherein the adverse cardiovascular outcome is developing CAD, an adverse effect of CAD, myocardial infarction (MI), or death.

9-10. (canceled)

11. The method of claim 1, wherein the subject is a patient known to have CAD or a patient suspected of having CAD, or
wherein the subject is a patient with significant CAD, stable CAD, insignificant CAD, or a recent acute coronary syndrome (ACS), or
wherein the subject has no symptoms of coronary artery disease, or has not had an acute myocardial infarction (AMI) event within the last 30 days, or
wherein the subject is a subject with an intermediate or high-risk result on a FRS test, coronary calcium test, or second-tier blood test.

12-17. (canceled)

18. A method of determining risk of an adverse cardiovascular outcome in a subject, the method comprising: comparing the level of each of a plurality of biomarkers in a test biological sample from the subject to a control level of the respective biomarker, wherein:
the plurality of biomarkers comprises (i) a fibrin and fibrinogen degradation product (FDP) marker, wherein the FDP marker includes a mixture of at least two fibrin and fibrinogen degradation products (FDPs) selected from the group consisting of fragment D, fragment E, D-dimer, fragment X, fragment Y, and an initial plasmin digest product (IPDP), and (ii) at least one inflammation or autoimmune disease biomarker; and
an increase in the levels of the plurality of biomarkers in the test sample compared to the control levels, indicates a risk of an adverse cardiovascular outcome in the subject.

19. The method of claim 18, wherein the control level of each biomarker is calculated from data comprising the levels of the biomarker in control biological samples from a plurality of control subjects, wherein the control subjects and the subject under assessment are of the same species and the test biological sample and the control samples comprise plasma or serum.

20. The method of claim 18, wherein the risk of the adverse cardiovascular outcome is increased if the levels in the test biological sample are higher than the control levels.

21. The method of claim 1, wherein the plurality of biomarkers further comprises an anti-cytomegalovirus antibody gene product or an antibody to Heat Shock Protein 60 (anti-HSP60) gene product.

22. The method of claim 1, wherein the FDP marker includes at least two FDPs, selected from the group consisting of fragment D, fragment E, and D-dimer or
wherein the FDP marker includes at least two FDPs selected from the group consisting of fragment X, fragment Y, and an IPDP.

23-24. (canceled)

25. The method of claim 1, wherein the test biological sample comprises:
(a) a body fluid or tissue from the subject; or
(b) one or more of: whole blood, blood fractions, blood components, plasma, platelets, serum, cerebrospinal fluid (CSF), bone marrow, urine, tears, milk, lymph fluid, organ tissue, nervous system tissue, non-nervous system tissue, muscle tissue, biopsy, necropsy, fat biopsy, fat tissue, cells, feces, placenta, spleen tissue, lymph tissue, pancreatic tissue, bronchoalveolar lavage (BAL), and synovial fluid.

26-27. (canceled)

28. The method of any of claim 1, wherein the measuring the level of the plurality of biomarkers in the test biological sample is carried out by immunoassay or an ELISA.

29. (canceled)

30. The method of claim 1, wherein the plurality of biomarkers include Heat Shock Protein 70 (HSP70), C-reactive protein (CRP), and the FDP marker.

31-33. (canceled)

34. The method of claim 1, wherein elevated levels of the plurality of biomarkers, in the aggregate, indicate an increased risk of the adverse cardiovascular outcome, or
wherein an elevated level of only one of the biomarkers, alone, would not indicate an increased risk of the adverse cardiovascular outcome.

35. The method of claim 34, wherein the level of the FDP marker is elevated if greater than 1 microgram per milliliter of sample.

36. The method of claim 34, wherein the plurality of biomarkers includes a CRP gene product and the level of the CRP gene product is elevated if greater than 3 milligrams per liter of sample.

37. The method of claim 34, wherein the plurality of biomarkers includes an HSP70 gene product and the level of the HSP70 gene product is elevated if detectable in the sample.

38. The method of claim 34, wherein elevated levels of the plurality of biomarkers, in the aggregate, indicate at least a 2 times greater risk, at least a 3 times greater risk, or at least a 5 times greater risk of acute myocardial infarction (AMI) or death, annually, in the subject, compared to a subject in which none of the plurality of biomarkers is elevated.

39-43. (canceled)

44. A method of treatment, comprising:
(a) assessing the risk of an adverse cardiovascular outcome in a subject by the method of claim 1; and
(b) treating the subject for the adverse cardiovascular outcome.

45. The method of claim 44, further comprising:
(c) repeating step (a) after a period of time following treatment, wherein a determination that levels of the biomarkers have not decreased or have not substantially decreased indicates that additional therapy is needed; and/or
(d) administering additional therapy to the subject.

46. (canceled)
